(19) United States
(12) Patent Application Publication
(10) Pub. No.: US 2023/0056502 A1
(43) Pub. Date: Feb. 23, 2023

(54) METHOD FOR DETERMINING THE ALLELE FREQUENCY/MUTATION RATE, AND DIAGNOSTICS

(71) Applicant: SensID GmbH, Rostock (DE)

(72) Inventors: Andreas BOLLMANN, Berlin (DE); Björn NOWACK, Seebad-Warnemünde (DE); Eileen STROH, Ribnitz (DE)

(21) Appl. No.: 17/792,726

(22) PCT Filed: Jan. 18, 2021
(86) PCT No.: PCT/EP2021/050962

§ 371 Date: Jul. 14, 2022

(30) Foreign Application Priority Data
Jan. 17, 2020 (EP) 20152341.2

(51) Int. Cl. C12Q 1/6886

(52) U.S. Cl. CPC C12Q 1/6886; C12Q 1/6858

(57) ABSTRACT

The present invention relates to a new method for determining the allele frequency and/or mutation rate in nucleic acids, in particular in tumor nucleic acids, in the context of a polymerase chain reaction (PCR), and to diagnostics for this purpose, wherein at least one reference nucleic acid (RN) and one mutation sequence with respect to the reference nucleic acid are used. This reference nucleic acid and mutation sequence allows polymerase chain reaction (PCR) methods to be validated, in particular on the basis of device parameters and sample preparation. Furthermore, the invention relates to an associated diagnosis and prognosis method, in particular for tumor diagnosis as part of a liquid biopsy.

The present invention relates to a new method for determining the allele frequency and/or mutation rate in nucleic acids, in particular in tumor nucleic acids, in the context of a polymerase chain reaction (PCR), and to diagnostics for this purpose, wherein at least one reference nucleic acid (RN) and one mutation sequence with respect to the reference nucleic acid are used. This reference nucleic acid and mutation sequence allows polymerase chain reaction (PCR) methods to be validated, in particular on the basis of device parameters and sample preparation. Furthermore, the invention relates to an associated diagnosis and prognosis method, in particular for tumor diagnosis as part of a liquid biopsy.

Current methods for detecting tumors, such as clinical-chemical tests in bodily fluids and tissue samples, or imaging methods, mean that malignant changes are often detected too late, despite all the progress in recent years. The main cause of the high mortality rate among tumor patients is not primary tumors developing, but metastases. In order to prevent tumors from metastasizing, any malignant changes need to be detected as early as possible. Furthermore, methods are needed which allow malignant cells to be correctly differentiated from normal cells. Both false-positive and false-negative results have fatal consequences for those affected. Methods are also required which provide tumor patients with an accurate prognosis and allow for treatment that is accordingly individually tailored to each patient.

Up to now, so-called tumor markers, in particular oncogenes, have been determined in bodily fluids such as blood, urine, sputum or tissue samples. These are components of tumor cells which are formed to be either enhanced or diminished and the changes thereto can be detected in bodily fluids such as blood, plasma, or serum. These tumor markers do not, however, allow for general screening for tumor diseases since their diagnostic specificities and sensitivities are often too low. Although these tumor markers were often able to indicate malignant changes at an earlier stage than imaging methods, they currently do not provide adequate detection of malignant changes.

The isolation, characterization and analysis of intracellular components, primarily nucleic acids (ribonucleic acids (RNA) and deoxyribonucleic acids (DNA)), is of vital importance for modern microbiology. Since the invention of the polymerase chain reaction (PCR) in 1983, a large number of nucleic-acid diagnostic methods have been developed which are used for detecting illnesses and pathogens, for example.

In the prior art, cell material of this kind is provided for diagnostics in the subsequent steps, such as sample preparation, extraction, concentration, isolation, purification, reverse transcription, amplification, and detection.

Owing to the highly effective amplification of tumor DNA by means of PCR, in particular “qPCR” (real-time quantitative polymerase chain reaction), “digital droplet PCR” (ddPCR), or “next generation sequencing” (NGS, or parallel sequencing, such as “massive parallel sequencing”), in recent years the so-called non-invasive liquid biopsy has become established in tumor diagnostics, in which circulating free DNA is tested in a sample. Circulating free DNA (cfDNA) of this kind can, for example, originate from a cancer/tumor cell (so-called ctDNA).

The average cfDNA quantity is higher in cancer patients than in healthy test subjects, and, in particular, the cfDNA plasma level in cancer patients is higher at an advanced stage than at a mild/early stage. ctDNA makes up e.g. 1% of the cfDNA (0.01-90% of the normal cfDNA, depending on the stage and location of the tumor).

There is, however, a great need for a standard, so that not only relative, but also absolute diagnostic information can be provided. The use of a standard of this kind is described in WO2018094183A1, for example.

Likewise, associated diagnostics are disclosed (claim 183).

Currently, both commercial tests and lab-developed tests (LDT) are available for mutations that need to be detected for tumor diagnostics. In-laboratory standards such as plasmid DNA or synthetic oligonucleotides, and many others, which constitute the prior art, are currently used as quality controls for validation or calibration.

It is, however, a drawback that, for the purposes of validation or calibration of a device or a method, these in-laboratory standards themselves have to be diluted and an automated process is not provided to do this, meaning that standardization is usually prone to errors. Furthermore, the stability of such dilutions is not guaranteed and, depending on the source, such as plasmid DNA from E. coli, there is the risk of RNA contamination. Therefore, the copy number determined by means of PCR may be incorrect in relative and absolute terms owing to a lack of adequate standardization.

Therefore, the standardization on the basis of a human reference nucleic acid needs further improvement, with a further mutation sequence being provided. The human reference nucleic acid and the mutation sequence form a standard.

Surprisingly, the inventors were able to establish that, with such a standard, the use of a human serum or plasma sample which is, however, free of DNA allows for improved validation and also diagnosis.

The problem addressed by the present invention is therefore to provide an improved method for detecting an allele frequency and/or mutation rate in a sample nucleic acid together with associated diagnosis, in which a suitable standard is intended to be used.

The problem is solved by a method for validating a polymerase chain reaction (PCR) method by means of determining the allele frequency and/or mutation rate in nucleic acids, comprising the steps of:

a.) providing at least one human reference nucleic acid (=wild type) and one human nucleic acid having one or more mutations (=mutation nucleic acid) to the reference nucleic acid in a DNA-free serum or plasma sample, wherein a particular allele frequency and/or mutation rate is predetermined,
and optionally
b.) providing a human reference nucleic acid (=wild type) from a.) in a DNA-free serum or plasma sample, wherein the allele frequency and/or mutation rate is 0%,
and optionally
c.) providing a DNA-free serum or plasma sample, wherein the particular allele frequency in a.) is detected, and where necessary is compared with b.) and/or c.).

The above method is referred to as the “validation method according to the invention” in the following.

A previously mentioned “particular allele frequency” is greater than or equal to 0% and can in particular take on values such as 0.01%, 0.1%, 0.5%, 1%, 2.5%, 5%, and many more.

Within the meaning of this invention, “reference nucleic acid” means an arbitrary and known human wild type sequence of an allele. Such alleles can be extracted from databases, such as COSMIC: https://cancer.sanger.ac.uk/cosmic, Targeted Cancer Care: http://targetedcancercare.massgeneral.org/My-Trial-Guide/Diseases/Lung-Cancer/KRAS/G12C-(c-34G-T).aspx, or OncoKB: https://oncokb.org/, My Cancer Genome: https://www.mycancergenome.org/.

In a preferred embodiment, the reference nucleic acid content is preferably 20-600 ng, in particular 400 ng DNA in serum or plasma, such as 400 ng/5 ml, equivalent to 80 ng/ml, equivalent to 0.08 ng/μl DNA in serum or plasma.

Furthermore, it is preferable for the reference nucleic acid to have a size of from 50 bp to 500 bp.

Within the meaning of this invention, “human nucleic acid having one or more mutations” or “mutation sequence or mutation nucleic acid” means such a sequence that has one or more mutations compared with a human wild type sequence of an allele as a reference nucleic acid. Here, known mutation sequences can be used or artificial mutations can be introduced in relation to the human wild type sequence. Methods for preparing such artificial mutations are described in the prior art. It is essential that, in the method according to the invention, this reference sequence and the mutation sequence are known (e.g. described on the basis of the nucleic acid sequence). Furthermore, it is preferable for the mutation sequence to have at least one tumor marker, e.g. oncogene, having known mutations. In addition, such tumor marker sequences or oncogenes can have further artificial mutations.

In a preferred embodiment, the content in a mutation sequence is less than the content in a reference sequence. Furthermore, it is preferable for the mutation sequence to have a size of from 50 bp to 500 bp.

Furthermore, it is preferable for the reference nucleic acid and mutation sequence to each be present in a particular concentration.

According to the invention, the allele frequency or mutation rate means the ratio of the reference nucleic acid to the mutation sequence (RN/MS), for example using the number of available copies of the reference nucleic acid to the mutation sequence or the ratio on the basis of the concentration of the reference nucleic acid to the mutation sequence.

For example, the allele frequency or mutation rate is 4.76% if 200 mutation sequences (“mutated allele”) to 4,000 copies of a reference nucleic acid (“wild type allele”) (here: 4.76%=(200×100%)/(200+4000)) are present together. Within the meaning of the allele frequency, this is therefore 4.76%, i.e. the frequency of the allele mutation or mutation sequence is 4.76% or the mutation rate is 4.76%.

In this way, validation samples according to the invention can be provided which have a particular or set allele frequency or mutation rate.

Within the meaning of this invention, validation means that, on the basis of the predetermined validation samples, the particular or set allele frequency or mutation rate can be detected as part of a polymerase chain reaction (PCR). This may be dependent both on device parameters and on the polymerase chain reaction (PCR) being carried out, and in particular on the sample preparation. The validation samples can be provided by means of a kit.

If the validation samples are not extracted in terms of their allele frequency/mutation rate when the polymerase chain reaction (PCR) is carried out, the sensitivity is not provided or the detection limit is too high, meaning that detection can take place. Furthermore, there is the possibility that, when preparing the validation samples for a PCR, too little DNA material could have been extracted. Therefore, the extraction efficiency may be defective, with consequential effects on the sample preparation with a parallel patient or test-subject sample of a sample nucleic acid.

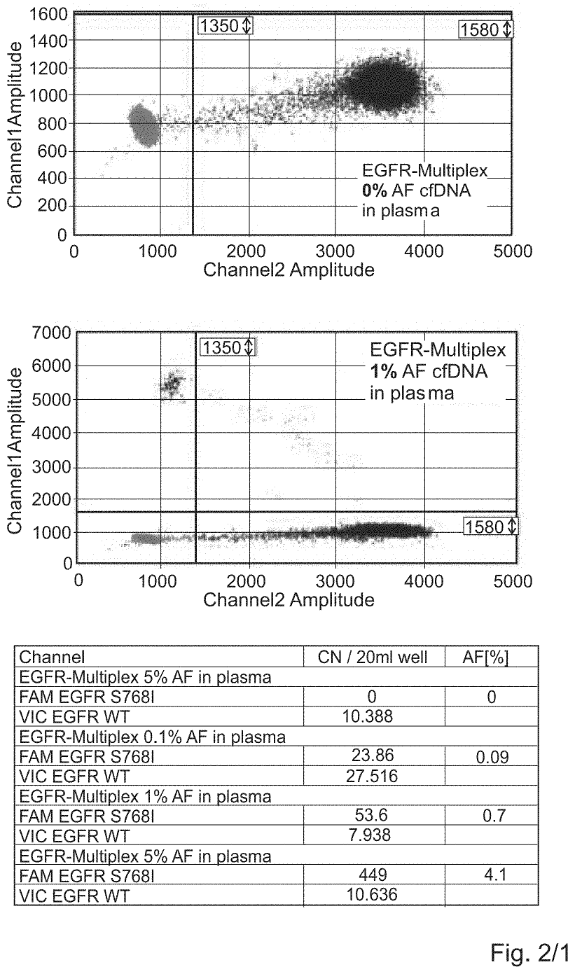

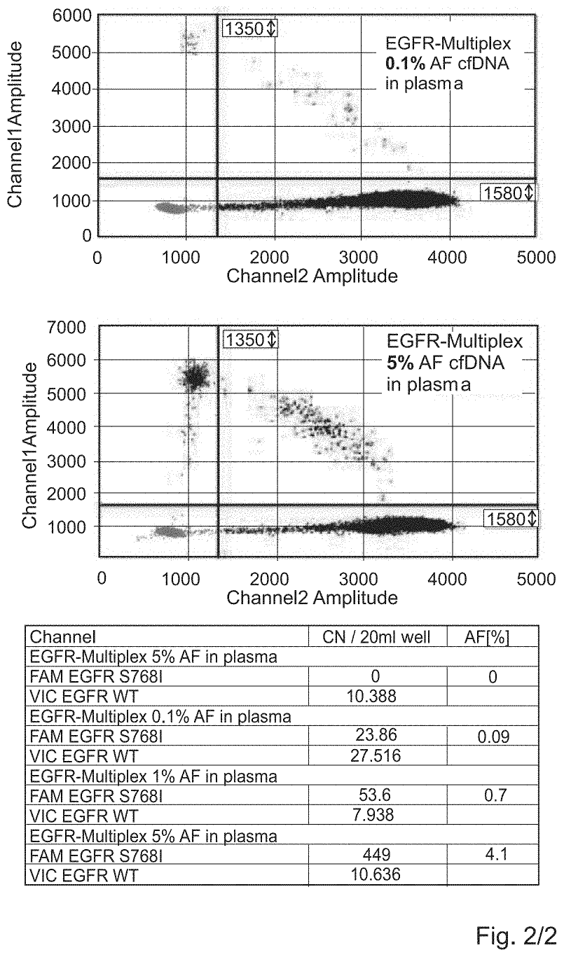

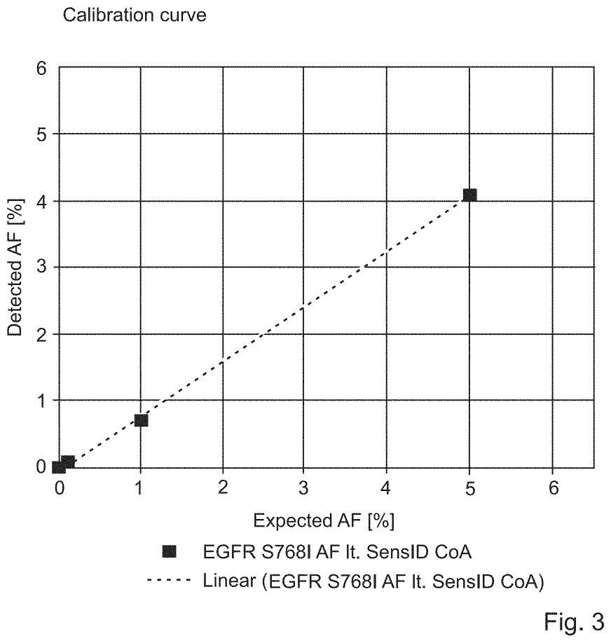

An exemplary validation is described in the examples and in FIGS. 2 and 3.

In another preferred embodiment, calibration or validation curves can be compiled.

An exemplary calibration curve is described in the examples and FIG. 3.

Surprisingly, in this way, advantageously low quantities of sample nucleic acid can be detected with adequate specificity and sensitivity. This allows for early information to be provided on the tumor activity, in particular the probability of metastasis. A preferred sample nucleic acid is cfDNA or ctDNA of a patient or test subject.

Therefore, the invention relates to a method for determining the allele frequency and/or mutation rate of at least one sample nucleic acid by means of a PCR method, in which the validation method according to the invention is carried out. Particularly advantageously, the sample nucleic acid can be quantitatively determined on the basis of the calibration or validation.

Another particular embodiment of the invention relates to a method for the diagnosis or prognosis of a tumor disease, wherein a change in the allele frequency and/or mutation rate of a sample nucleic acid from a first sample and a second and/or further sample allows for early detection and detection, for the degree of severity to be assessed, and for progression to be assessed accompanied by treatment, wherein calibration is carried out by means of the validation method according to the invention.

In particular, the second or further sample can be taken from a patient at a later point in time.

Particularly advantageously, providing a DNA-free serum or plasma sample makes it possible to validate the detection limit of the standard nucleic acid and of the sample nucleic acids. A particularly advantageous application therefore relates to the validation of devices for carrying out a polymerase chain reaction (PCR), in particular next generation sequencing (NGS), wherein the method according to the invention is carried out.

Particularly advantageously, in this way, a precisely predetermined quantity of DNA material or concentration of the standard according to the invention can be introduced into a sample, with this sample largely simulating a patient serum or plasma sample and being highly suitable for carrying out tumor diagnostics.

Furthermore, it is preferable for the concentration of mutation nucleic acid to preferably be 4×10E-05 fg/μl to 0.03 fg/μl.

The mutation sequence according to the invention preferably comprises a tumor marker, in particular an oncogene selected from the group of MTOR, MPL, NRAS, PARP1, AKT3, DNMT3A, MSH2, IDH1, VHL, MLH1, MYD88, CTNNB1, ATR, PIK3CA, FGFR3, PDGFRA, KIT, FBXW7, APC, GABRG2, NPM1, EGFR, MET, BRAF, EZH2, JAK2, GNAQ, RET, PTEN, ATM, KRAS, PTPNII, FLT3, RB1, PARP2, ARHGAP5, AKT1, RAD51, IDH2, TP53, NF1, SMAD4, AKT2, ERCC1, GNAS, ERBB2, FOXL2, NOTCH1, or NTKR.

TABLE 1
oncogenes (see e.g. “COSMIC” (supra)):

| Genes | Position | REF | ALT | Strand | CDS | AA | COSID |
|---|---|---|---|---|---|---|---|
| MTOR | 11291097 | T | A | − | 2664 A > T | L888F | COSM94356 |
| MPL | 43815009 | G | T | + | 1544 G > T | W515L | COSM18918 |
| NRAS | 115256529 | T | C | − | 182 A > G | Q61R | COSM584 |
| PARPI | 226551691 | TC | T | − | 2738 | G913fs*4 | COSM21691 |
| AKT3 | 243809253 | T | A | − | 371 A > T | Q124L | COSM48227 |
| DNMT3A | 25457243 | G | A | − | 2644 C > T | R882C | COSM53042 |
| MSH2 | 47705449 | TG | T | + | 2250 delG | G751fs*12 | COSM111644 |
| MSH2 | 47705558 | ACT | A | + | 2359 2360 | L787fs* 11 | COSM26122 |
| | | | | | delCT | | |
| IDHI | 209113113 | G | A | − | 394C > T | R132C | COSM28747 |
| VHL | 10188282 | TIGAC | T | + | 426 429 | G144fs*14 | COSM18578 |
| | | | | | del TGAC | | |
| MLHI | 37067240 | T | A | + | 1151 T > A | V384D | COSM26085 |
| MVD88 | 38182641 | T | c | + | 794 T > C | L265P | COSM85940 |
| CTNNBI | 41266124 | A | G | + | 121 A > G | T41A | COSM5664 |
| ATR | 142254972 | GCTITIAT | G | − | 3790 3796 | 11264fs*24 | COSM20627 |
| | | | | | del ATAAAAG | | |
| PIK3CA | 179218303 | G | A | + | 1633 G > A | E545K | COSM763 |
| PIK3CA | 179218304 | A | C | | 1634A > C | E545A | COSM12458 |
| PIK3CA | 179218305 | G | T | | 1635G > T | E545D | COSM765 |
| PIK3CA | 179218304 | A | G | | 1634A > G | E545G | COSM764 |
| PIK3CA | 179218294 | G | A | | 1624G > A | E542K | COSM760 |
| PIK3CA | 179210192 | T | C | | 1258T > C | C420R | COSM757 |
| PIK3CA | 179218306 | C | G | | 1636C > G | Q546E | COSM6147 |
| PIK3CA | 179218307 | A | G | | 1637A > G | Q546R | COSM12459 |
| PIK3CA | 179234297 | A | G | + | 3140 A > G | H1047R | COSM775 |
| PIK3CA | 179234297 | A | T | | 3140A > T | H1047L | COSM776 |
| PIK3CA | 179234296 | C | T | | 3139C > T | H1047Y | COSM774 |
| PIK3CA | 178952149 | c | CA | + | 3204 3205 ins A | N1068fs*4 | COSM12464 |
| FGFR3 | 1803568 | c | G | + | 746 C > G | S249C | COSM715 |
| PDGFRA | 55141048 | T | TA | + | 1694 1695 ins A | S566fs*6 | COSM28053 |
| PDGFRA | 55152093 | A | T | + | 2525 A > T | D842V | COSM736 |
| KIT | 55599321 | A | T | + | 2447 A > T | D816V | COSM1314 |
| FBXW7 | 153249384 | c | T | − | 1394 G > A | R465H | COSM22965 |
| APC | 112175538 | GC | G | + | 4248 del C | 11417fs*2 | COSM18584 |
| APC | 112175639 | c | T | + | 4348 C > T | R1450* | COSM13127 |
| APC | 112175957 | A | AA | + | 4666 4667 ins A | T1556fs*3 | COSM18561 |
| GABRA6 | 161117296 | G | c | + | 763 G > C | V255L | COSM70853 |
| GABRG2 | 161580301 | A | G | + | 1355 A > G | Y452C | COSM74722 |
| NPM | 170837547 | G | GTCTG | + | 863 864 | W288fs*12 | COSM17559 |
| | | | | | ins TCTG | | |
| EGFR | 55174787 | GGAATI- | G | + | 2236 2250 | E746 A750 | COSM6225 |
| | | AAGAG- | | | del 15 | del ELREA | |
| | | AAGCA | | | | | |
| EGFR | 55249012 | c | CGGT | + | 2310 2311 | 0770 N771 | COSM12378 |
| | | | | | ins GGT | ins G | |
| EGFR | 55181378 | c | T | + | 2369 C > T | T790M | COSM6240 |
| EGFR | 55191822 | T | G | + | 2573 T > G | L858R | COSM6224 |
| EGFR | 55174014 | G | A | | 2155G > A | G719S | COSM6252 |
| EGFR | 55191831 | T | A | | 2582T > A | L861Q | COSM6213 |
| EGFR | 55181312 . . . | | insTGTGGCCAG | | 2303_ | V769_ | COSM20884 |
| | 55181313 | | | | 2304insTG- | D770ins | |
| | | | | | TGGCCAG | ASV | |
| EGFR | 55174791 . . . | | delTCTCCGAAAGCCAA | | 2254_ | S752_ | COSM6256 |
| | 55174814 | | CAAGGAAATC | | 2277del24 | l759delS | |
| | | | | | | PKANKEI | |
| EGFR | 55181312 | G | T | | c.2303G > T | S768I | COSM6241 |
| MET | 116423428 | T | G | + | 3757 T > G | Y1253D | COSM700 |
| BRAF | 140753336 | T | A | − | 1799 T > A | V600E | COSM476 |
| EZH2 | 148508727 | T | A | − | 1937 A > T | V646F | COSM37028 |
| JAK2 | 5073770 | G | T | + | 1849 G > T | V617F | COSM12600 |
| GNAQ | 80409488 | T | G | − | 626 A > C | Q209P | COSM28758 |
| RET | 43617416 | T | c | + | 2753 T > C | M918T | COSM965 |
| PTEN | 89692904 | c | T | + | 388 C > T | R130* | COSM5152 |
| PTEN | 89717716 | A | AA | + | 741 742 ins A | P248fs*5 | COSM4986 |
| PTEN | 89717774 | AA | A | + | 800 del A | K267fs*9 | COSM5809 |
| ATM | 108117846 | TGT | T | + | 1058 1059 | C353fs*5 | COSM21924 |
| | | | | | del GT | | |
| ATM | 108175462 | G | A | + | 5557 G > A | D1853N | COSM41596 |
| KRAS | 25245350 | c | T | − | 35 G > A | G12D | COSM521 |
| KRAS | 25380275 | A | T | | c.183A > T | Q61H | COSM555 |
| KRAS | 25227343 | C | A | | c.181C > A | Q61K | COSM549 |
| KRAS | 25225628 | G | A | | 436G > A | A146T | COSM19404 |
| PTPNII | 112888210 | G | A | + | 226 G > A | E76K | COSM13000 |
| FLT3 | 28592642 | c | A | − | 2503 G > T | D835V | COSM783 |
| RBI | 48941648 | c | T | + | 958 C > T | R320* | COSM891 |
| PARP2 | 20820412 | A | c | + | 398 A > C | D133A | COSM75849 |
| ARHGAP5 | 32561739 | G | A | + | 1864 G > A | E622K | COSM88502 |
| AKTI | 105246455 | c | T | − | 145 G > A | E49K | COSM36918 |
| AKTI | 104780214 | c | T | − | 49 G > A | E17K | COSM33765 |
| RAD51 | 41001312 | c | T | + | 433 C > T | Q145* | COSM117943 |
| IDH2 | 90631838 | c | T | − | 515 G > A | R172K | COSM33733 |
| IDH2 | 90631934 | c | T | − | 419 G > A | R140Q | COSM41590 |
| TP53 | 7577120 | c | T | − | 818 G > A | R273H | COSM10660 |
| TP53 | 7577538 | c | T | − | 743 G > A | R248Q | COSM10662 |
| TP53 | 7577557 | AG | A | − | 723 del C | C242fs*5 | COSM6530 |
| TP53 | 7578406 | c | T | − | 524 G > A | R175H | COSM10648 |
| TP53 | 7579423 | GG | G | − | 263 del C | S90fs*33 | COSM18610 |
| NFI | 29556989 | T | TAC | + | 2987 2988 | R997fs*16 | COSM41820 |
| | | | | | ins AC | | |
| NFI | 29576111 | c | T | + | 4084 C > T | R1362* | COSM24443 |
| NFI | 29679317 | TG | T | + | 7501 del G | E2501fs*22 | COSM24468 |
| SMAD4 | 48603093 | T | TT | + | 1394 1395 ins T | A466fs*28 | COSM14105 |
| AKT2 | 40761084 | c | A | − | 268 G > T | V90L | COSM93894 |
| ERCCI | 45924470 | G | T | − | 287 C > A | A96E | COSM140843 |
| GNAS | 57484420 | c | T | + | 601 C > T | R201C | COSM27887 |
| ERBB2 | 39724728 . . . | | insGCATACGTGATG | | 2310_2311ins12 | p.E770_ | COSM404915 |
| | 39724729 | | | | | A771in | |
| | | | | | | sAYVM | |

Within the meaning of this invention, “diagnosis” means that a conclusion can be drawn on the tumor activity from the mutation rate. The greater the mutation rate, the greater the tumor activity, in particular the probability of metastasis. Therefore, the invention relates to the diagnosis of cancer or a tumor. The term “diagnosis” covers the medical diagnostics and related tests, in particular in vitro diagnostics and laboratory diagnostics. Preferably, the information relates to an illness or a condition of a patient.

In the context of this invention, “patient” is understood to be any test subject.

Further subject matter of the invention relates to a kit containing

a.) part (e.g. tube) having at least one human reference nucleic acid and one nucleic acid having one or more mutations relative to the reference sequence in a DNA-free serum or plasma sample, wherein a predetermined allele frequency is set,
and optionally
b.) part having a human reference nucleic acid in a DNA-free serum or plasma sample,
and optionally
c.) part having a DNA-free serum or plasma sample,
for carrying out one of the above-described methods or the use of a kit of this kind for carrying out one of the above-described methods.

In the following, the invention is explained using examples. However, the invention is not limited to the examples, but instead is universally applicable in principle.

### EXAMPLE 1

Carrying out the method according to the invention by providing a kit:

1) Extraction of nucleic acids (DNA), in particular cfDNA, from the serum or plasma reference material according to the invention using commercial kits, such as the QIAamp ccfDNA/RNA kit (Qiagen®), the PME Free-Circulating DNA Extraction Kit (AnalytikJena®), or the MagMAX™ Cell-Free DNA Isolation Kit (ThermoFisher®)).
2) Quantification of the nucleic acids, in particular cfDNA in the eluate fluorometrically e.g. using Qubit® (ThermoFisher®) or spectrophotometrically using NanoDrop® or another spectrometer.
3) Optionally, a qualitative analysis of the nucleic acids, in particular cfDNA, can be carried out, such as a fragment length analysis using a bioanalyzer (Agilent®), fragment analyzer (Agilent®)), or pulsed-field gel electrophoresis.
4) The obtained nucleic acid, in particular cfDNA, is then supplied to a PCR.

At the same time, a corresponding serum or plasma sample from a patient can be prepared.

Output ddPCR:

Using the example of a QX200 ddPCR system from BioRad®:

By means of ddPCR, a defined volume of the nucleic acid to be tested is fractionated into thousands of individual reaction chambers. In this process, the DNA sequences to be tested are fractionated into these reaction chambers using Poisson distribution. Amplification of the nucleic acids takes place in the reaction chambers if the nucleic acid is present. The mutation sequence triggers a PCR reaction that can be differentiated in color from the reference sequence (wild type sequence).

By means of relevant software, the results can be evaluated and represented in a plot, in particular a 2D plot of amplitude:

The results detected in the FAM channel (blue fluorescence) (channel 1 amplitude) are plotted on the Y axis.

The results detected in the HEX/VIC channel (green fluorescence) (channel 2 amplitude) are plotted on the X axis.

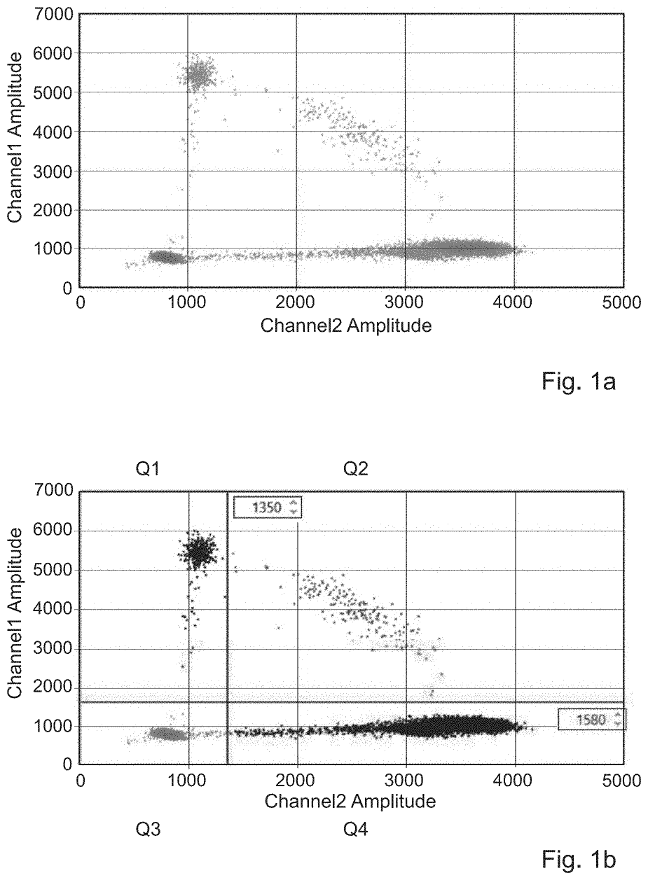

Each plotted point represents a reaction chamber by a reaction having been carried out (FIG. 1a) with a reference sequence (wild type sequence) (Q4), a mutation sequence (Q1), with both sequences (Q2), or with no sequence (Q4), because no target nucleic acid was present, the obtained point clouds Q1 to Q4 being shown in a graph in FIG. 1b.

An evaluation takes place such that the raw data (plotted points) determined by the software provide numbers quantified in an absolute manner for the reference sequence and the mutation sequence.

Table 1: Example values for Q1 and Q4:

Well Data

| Well | DyeName (s) | Copies/20 μWell |
|---|---|---|
| C11 | FAM | 477 |
| C11 | VIC | 10632 |

The allele frequency can be determined from these values, e.g. manually or using software, i.e. the copy numbers for the reference sequence and the mutation sequence are calculated in accordance with the following formula for the allele frequency:

(CN_{Mut}*100%)/(CN_{Mut}+CN_{Wt})=VAF or MAF

CN_{Mut}=copy numbers of mutation sequence
CN_{wt}=copy numbers of reference sequence
VAF=variation in allele frequency or MAF=mutation in allele frequency.

In this way, the tubes of the kit (relevant standard sample with preset allele frequency, such as 0.0, 0.1, 1, and 5% in FIG. 2) undergo the method according to the invention, with the following information advantageously being obtained:

1) Qualitative: is the mutation detectable in the mutation sequence, yes/no?
2) Copy numbers (“CN”) quantified in an absolute manner for the reference sequence and mutation sequence,
3) Determining the allele frequency.

Examples are set out in FIG. 2.

A calibration curve can be compiled from the calculated values (FIG. 2, table) and the validation can be completed (FIG. 3).

Simple evaluation is also possible by means of NGS, since each copy corresponds to a “read,” and the reference sequence and mutation sequence can likewise be differentiated in a simple manner.

Patient or test-subject samples (supra) can be determined at the same time or different times on the basis of the calibration curve and a diagnosis can be made.

1.-13. (canceled)

14. Method for validating a polymerase chain reaction (PCR) method by means of determining the allele frequency and/or mutation rate in nucleic acids, comprising the steps of:
a.) providing at least one human reference nucleic acid and one human nucleic acid having one or more mutations relative to the reference nucleic acid in a DNA-free serum or plasma sample, wherein a particular allele frequency and/or mutation rate is predetermined,
and optionally
b.) providing a human reference nucleic acid from a.) in a DNA-free serum or plasma sample,
and optionally
c.) providing a DNA-free serum or plasma sample,
wherein the particular allele frequency in a.) is detected, and where necessary is compared with b.) and/or c.).

15. Method for validating a PCR method by means of determining the allele frequency and/or mutation rate in nucleic acids according to claim 14, characterized in that the method is carried out for a plurality of predetermined allele frequencies from a.).

16. Method for validating a PCR method by means of determining the allele frequency and/or mutation rate in nucleic acids according to claim 15, characterized in that a validation and calibration curve is obtained.

17. Method for validating a PCR method for determining the allele frequency and/or mutation rate in nucleic acids according to claim 14, characterized in that the concentrations of reference nucleic acid and mutation nucleic acids in a.) and b.) are predetermined.

18. Method for validating a PCR method for determining the allele frequency and/or mutation rate in nucleic acids according to claim 14, characterized in that the mutation nucleic acids comprise at least one tumor marker.

19. Method for validating a PCR method for determining the allele frequency and/or mutation rate in nucleic acids according to claim 14, characterized in that a qPCR (real-time quantitative polymerase chain reaction), digital droplet PCR (ddPCR), or “next generation sequencing” (NGS) is carried out.

20. Method for determining the allele frequency and/or mutation rate of at least one sample nucleic acid by means of a PCR method, wherein calibration is carried out by means of a method according to claim 14.

21. Method for determining the allele frequency and/or mutation rate of at least one sample nucleic acid by means of a PCR method, wherein the sample nucleic acid is quantitatively determined.

22. Method for determining the allele frequency and/or mutation rate of at least one sample nucleic acid by means of a PCR method according to claim 20, characterized in that the sample nucleic acid of a patient is a cfDNA or ctDNA.

23. Method for the diagnosis or prognosis of a tumor disease, wherein a change in the allele frequency and/or mutation rate of a sample nucleic acid from a first sample and a second and/or further sample allows for early detection and detection, for the degree of severity to be assessed, and for progression to be assessed accompanied by treatment, wherein calibration is carried out by means of a method according to claim 14.

24. Method for the diagnosis or prognosis of a tumor disease according to claim 23, wherein the second or further sample is taken from a patient at a later point in time.

25. Kit containing
a.) part having at least one human reference nucleic acid and one nucleic acid having one or more mutations relative to the reference sequence in a DNA-free serum or plasma sample, wherein a predetermined allele frequency is set,
and optionally
b.) part having a human reference nucleic acid in a DNA-free serum or plasma sample,
and optionally
c.) part having a DNA-free serum or plasma sample,
for carrying out a method according to claim 14.
